(19) United States
(12) Patent Application Publication
(10) Pub. No.: US 2006/0058293 A1
(43) Pub. Date: Mar. 16, 2006

(54) Combination of bupropion and a second compound for affecting weight loss

(76) Inventors: Eckard Weber, San Diego, CA (US); Michael Alexander Cowley, Portland, OR (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/194,201

(22) Filed: Aug. 1, 2005

Related U.S. Application Data
(60) Provisional application No. 60/598,558, filed on Aug. 3, 2004.

(51) Int. Cl. A61K 31/5377; A61K 31/454; A61K 31/415; A61K 31/195; A61K 31/137

(52) U.S. Cl. 514/232.5; 514/406; 514/326; 514/469; 514/567; 514/649

(57) ABSTRACT

Disclosed are compositions for affecting weight loss comprising bupropion and a second compound, where the second compound causes increased agonism of a melanocortin 3 receptor (MC3-R) or a melanocortin 4 receptor (MC4-R) compared to normal physiological conditions, antagonizes cannabinoid receptor activity, or is useful in the treatment of bipolar disorders. Also disclosed are methods of affecting weight loss, increasing energy expenditure, increasing satiety in an individual, or suppressing the appetite of an individual, comprising identifying an individual in need thereof and treating that individual with a combination of bupropion and a compound that enhances α-MSH activity, antagonizes cannabinoid receptor activity, or is useful in the treatment of bipolar disorders.

### RELATED APPLICATIONS

This application claims priority under 35 U.S.C. § 119(e) to the U.S. Provisional Patent Application Ser. No. 60/598,558, filed on Aug. 3, 2004, by Weber et al., and entitled “COMBINATION OF BUPROPION AND A SECOND COMPOUND FOR AFFECTING WEIGHT LOSS,” the entire disclosure of which is hereby incorporated by reference herein in its entirety.

### BACKGROUND OF THE INVENTION

1. Field of the Invention

The present invention is in the field of pharmaceutical compositions and methods for the treatment of obesity and for affecting weight loss in individuals.

2. Description of the Related Art

Obesity is a disorder characterized by the accumulation of excess fat in the body. Obesity has been recognized as one of the leading causes of disease and is emerging as a global problem. Increased instances of complications such as hypertension, non-insulin dependent diabetes mellitus, arteriosclerosis, dyslipidemia, certain forms of cancer, sleep apnea, and osteoarthritis have been related to increased instances of obesity in the general population.

Obesity has been defined in terms of body mass index (BMI). BMI is calculated as weight (kg)/[height (m)]². According to the guidelines of the U.S. Centers for Disease Control and Prevention (CDC), and the World Health Organization (WHO) (World Health Organization. Physical status: The use and interpretation of anthropometry. Geneva, Switzerland: World Health Organization 1995. WHO Technical Report Series), for adults over 20 years old, BMI falls into one of these categories: below 18.5 is considered underweight, 18.5-24.9 is considered normal, 25.0-29.9 is considered overweight, and 30.0 and above is considered obese.

Prior to 1994, obesity was generally considered a psychological problem. The discovery of the adipostatic hormone leptin in 1994 (Zhang et al., “Positional cloning of the mouse obese gene and its human homologue,” Nature 1994; 372:425-432) brought forth the realization that, in certain cases, obesity may have a biochemical basis. A corollary to this realization was the idea that the treatment of obesity may be achieved by chemical approaches. Since then, a number of such chemical treatments have entered the market. The most famous of these attempts was the introduction of Fen-Phen, a combination of fenfluramine and phentermine. Unfortunately, it was discovered that fenfluramine caused heart-valve complications, which in some cases resulted in the death of the user. Fenfluramine has since been withdrawn from the market. There has been some limited success with other combination therapy approaches, particularly in the field of psychological eating disorders. One such example is Devlin, et al., Int. J. Eating Disord. 28:325-332, 2000, in which a combination of phentermine and fluoxetine showed some efficacy in the treatment of binge eating disorders. Of course, this disorder is an issue for only a small portion of the population.

In addition to those individuals who satisfy a strict definition of medical obesity, a significant portion of the adult population is overweight. These overweight individuals would also benefit from the availability of an effective weight-loss composition. Therefore, there is an unmet need in the art to provide pharmaceutical compositions that can affect weight loss without having other adverse side effects.

### SUMMARY OF THE INVENTION

Disclosed are compositions for affecting weight loss comprising bupropion, or a metabolite thereof, and a second compound, where the second compound causes increased agonism of a melanocortin 3 receptor (MC3-R) or a melanocortin 4 receptor (MC4-R) compared to normal physiological conditions or causes antagonism of a cannabinoid receptor activity.

Also disclosed are methods of affecting weight loss, increasing energy expenditure, increasing satiety in an individual, or suppressing the appetite of an individual, comprising identifying an individual in need thereof and treating that individual with a combination of bupropion, or a metabolite thereof, and a compound that enhances α-MSH activity or antagonizes cannabinoid receptor activity.

### DETAILED DESCRIPTION OF THE PREFERRED EMBODIMENTS

Arcuate nucleus neurons are known to be responsive to a wide array of hormones and nutrients, including leptin, insulin, gonadal steroids, and glucose. In addition to potential transport mechanisms, peripheral substances may access these neurons via arcuate cell bodies in and projections to the median eminence, a region considered to be a circumventricular organ, which lacks a blood-brain barrier. Cone et al., “The arcuate nucleus as a conduit for diverse signals relevant to energy homeostasis,” Int'l Journal of Obesity (2001) 25, Suppl 5, S63-S67.

Administration of exogenous leptin activates a number of different neurons in hypothalamic and brainstem cell groups that bear leptin receptor. Leptin-responsive neurons in the arcuate nucleus include both those containing neuropeptide Y (NPY) and agouti-related peptide (AgRP) in the medial part of the nucleus and those containing both pro-opiomelanocortin (POMC) and its derivatives, including α-melanocyte stimulating hormone (α-MSH), as well as cocaine and amphetamine-related transcript (CART). Saper et al., “The need to feed: Homeostatic and hedonic control of eating,” Neuron, 36:199-211 (2002).

The leptin-responsive POMC neurons in the arcuate nucleus are thought to cause anorexia and weight reduction by means of the action of α-MSH on melanocortin 3 and/or 4 receptors (MC3-R, MC4-R). The highest MC3-R expression level is in the hypothalamus and limbic system, whereas MC4-R mRNA is expressed in virtually all major brain regions. Some of the metabolic effects resulting from stimulation of MC4-R are decreased food intake and an increase in energy expenditure through stimulation of thyrotropin-releasing hormone and activation of the sympathetic nervous system. Targeted deletion of the MC4-R gene produces obesity, hyperphagia, hyperinsulinemia, and reduced energy expenditure. Targeted deletion of MC3-R results in increased adiposity due to decreased energy expenditure. Korner et al., “The emerging science of body weight regulation and its impact on obesity treatment,” J. Clin. Invest. 111(5):565-570 (2003). Thus, increased concentrations of α-MSH in the central nervous system (CNS) increase its action on MC3-R and/or MC4-R and result in a suppressed appetite.

POMC neurons also release β-endorphin when they release α-MSH. β-endorphin is an endogenous agonist of the μ-opioid receptors (MOP-R), found on the POMC neurons. Stimulation of MOP-R decreases the release of α-MSH. This is a biofeedback mechanism that under normal physiological conditions controls the concentration of α-MSH in the CNS. Thus, blocking MOP-R by opioid antagonists will break the feedback mechanism, which results in continued secretion of α-MSH and an increase in its concentration in the CNS.

A second population of neurons in the arcuate nucleus tonically inhibits the POMC neurons. These POMC-inhibiting neurons secrete NPY, the neurotransmitter γ-aminobutyric acid (GABA), and AgRP. NPY and GABA inhibit POMC neurons, via NPY Y1 receptors and GABA receptors, respectivley. Thus, within the arcuate nucleus NPY and GABA inhibit the release of α-MSH, and therefore are stimulators of feeding. It is known that leptin inhibits the release of GABA from NPY terminals synapsing onto POMC neurons, whereas ghrelin, an orexigenic peptide, stimulates the ghrelin receptors on NPY neurons and increase the secretion of NPY and GABA onto the POMC cells, which in turn inhibits the release of α-MSH.

AgRP stimulates food intake in the rat through antagonism of the interaction of α-MSH at MC4-R. Expression of the AgRP gene is suppressed by leptin.

Serotonin, also known as 5-hydroxytryptamine or 5-HT, activates the POMC neurons to secrete α-MSH. However, serotonin is taken up and removed from action by specific transporters so that a single serotonin molecule has short term effects. It is known that selective serotonin re-uptake inhibitors (SSRIs) prevent the uptake of serotonin and increase its concentrations in the CNS. Thus, SSRIs also increase the secretion of α-MSH and its concentrations in the CNS.

Dopamine also increases the activity of POMC neurons to secrete α-MSH. Like serotonin, dopamine is also taken up and removed from action so that a single dopamine molecule has short term effect. Dopamine re-uptake inhibitors, which prevent or reduce the uptake of dopamine, can also increase the secretion of α-MSH and its concentrations in the CNS.

Therefore, increased secretion of α-MSH through various mechanisms, such as serotonin re-uptake inhibition, are among the strategies that the methods and pharmaceutical compositions of the present invention pursue in order to produce a biochemical anorexigenic effect.

The present invention provides a multi-faceted combination therapy approach to the problem of weight loss. It addresses not just single molecules, messengers, or receptors, but instead acts on multiple points in the feeding and satiety pathway. Aspects of the present invention are directed to increasing the concentrations of α-MSH in the CNS by stimulating the release of α-MSH, suppressing its metabolism, reducing the antagonism of its interaction at MC3/4-R, and suppressing any feedback mechanisms that slow or stop its release. Aspects of the present invention include pharmaceutical compositions whose components achieve one or more of these functions. The present inventors have discovered that a combination of two or more of the compounds disclosed herein results in a synergistic effect that affects weight loss more quickly and on a more permanent basis.

Thus, in a first aspect, the present invention is directed to a composition for the treatment of obesity or for affecting weight loss comprising bupropion, or a metabolite thereof, or a pharmaceutically acceptable salt or prodrug thereof, and a second compound, where the second compound causes increased agonism of a melanocortin 3 receptor (MC3-R) or a melanocortin 4 receptor (MC4-R) compared to normal physiological conditions.

In another aspect, the present invention is directed to a composition for the treatment of obesity or for affecting weight loss comprising bupropion, or a metabolite thereof, or a pharmaceutically acceptable salt or prodrug thereof, and a second compound, where the second compound is a cannabinoid receptor antagonist.

In yet another aspect, the the present invention is directed to a composition for the treatment of obesity or for affecting weight loss comprising bupropion, or a metabolite thereof, or a pharmaceutically acceptable salt or prodrug thereof, and a second compound, where the second compound is an agent useful in the treatment of bipolar disorders.

In some embodiments, the second compound is not a compound that causes increased agonism of a melanocortin 3 receptor (MC3-R) or a melanocortin 4 receptor (MC4-R) compared to normal physiological conditions, while in other embodiments, the second compound is not a cannabinoid receptor antagonist.

Bupropion, whose chemical name is (±)-1-(3-chlorophenyl)-2-[(1,1-dimethylethyl)amino]-1-propanone, is the active ingredient in the drugs marketed as ZYBAN® and WELLBUTRIN®, and is usually administered as a hydrochloride salt. Throughout the present disclosure, whenever the term “bupropion” is used, it is understood that the term encompasses bupropion as a free base, or as a physiologically acceptable salt thereof. Bupropion may be administered orally as 75 mg or 100 mg tablets, or as 100 mg or 150 mg tablets in a sustained release formulation. Preparing tablets containing other dosages of bupropion is well within the skill of those of ordinary skill in the art.

The metabolites of bupropion suitable for inclusion in the methods and compositions disclosed herein include the erythro- and threo-amino alcohols of bupropion, the erythro-amino diol of bupropion, and morpholinol metabolites of bupropion. In some embodiments, the metabolite of bupropion is (±)-(2R*,3R*)-2-(3-chlorophenyl)-3,5,5-trimethyl-2-morpholinol. In some embodiments the metabolite is (−)-(2R*,3R*)-2-(3-chlorophenyl)-3,5,5-trimethyl-2-morpholinol, while in other embodiments, the metabolite is (+)-(2S,3S)-2-(3-chlorophenyl)-3,5,5-trimethyl-2-morpholinol. Preferably, the metabolite of bupropion is (+)-(2S,3S)-2-(3-chlorophenyl)-3,5,5-trimethyl-2-morpholinol, which is known by its common name of radafaxine. The scope of the present invention includes the above-mentioned metabolites of bupropion as a free base, or as a physiologically acceptable salt thereof.

In certain embodiments, the second compound causes increased activity of the POMC neurons, leading to greater agonism at MC3-R and/or MC4-R.

In certain embodiments compositions and the methods described herein cause weight loss in a mammal. The mammal may be selected from the group consisting of mice, rats, rabbits, guinea pigs, dogs, cats, sheep, goats, cows, primates, such as monkeys, chimpanzees, and apes, and humans.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts thereof with amino acids such as arginine, lysine, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

In certain embodiments, the second compound in the pharmaceutical compositions of the present invention triggers the release of α-melanocyte stimulating hormone ((α-MSH). The second compound may increase the extracellular serotonin concentrations in the hypothalamus. In some embodiments, the second compound is selected from the group consisting of a selective serotonin reuptake inhibitor (SSRI), a serotonin 2C agonist, and a serotonin 1B agonist. In further embodiments, the second compound is selected, e.g., from the group consisting of fluoxetine, fluvoxamine, sertraline, paroxetine, citalopram, escitalopram, sibutramine, duloxetine, and venlafaxine, and a pharmaceutically acceptable salt or prodrug thereof.

The terms “serotonin 1B receptor,” “serotonin 2C receptor,” “5-HT1b receptor,” and “5-HT2c receptor” refer to receptors found more commonly in rodents. It is understood by those of skill in the art that other mammals have serotonin receptors on various neurons that are analogous in function and form to these receptors. Agonists or antagonists at these non-rodent, preferably human, serotonin receptors are within the scope of the present invention.

In certain embodiments, the second compound suppresses the expression of the AgRP gene or the production or release of agouti-related protein (AgRP). In some of these embodiments, the second compound suppresses the activity of neurons that express AgRP.

In other embodiments, the second compound suppresses the expression of the NPY gene or the production or release of neuropeptide Y (NPY). In some of these embodiments, the second compound suppresses the activity of neurons that express NPY. In further embodiments, the second compound is selected from the group consisting of NPY antagonists, ghrelin antagonists, and leptin. In certain other embodiments, the second compound agonizes NPY Y2 receptor.

In some embodiments, the second compound is an NPY receptor antagonist. In certain embodiments, the receptor is NPY Y1, while in other embodiments the receptor is NPY Y5. In some embodiments, the NPY receptor antagonist is S-2367, a compound developed by Shionogi Co. Ltd. of Japan.

In certain embodiments, the second compound is selected from α-MSH, melanotan, MT II (melanotan II, disclosed in U.S. Pat. No. 5,674,839, which is hereby incorporated by reference in its entirety), PT141 (developed by Palatin Technologies), the cyclic peptide Maltose Binding Peptide 10 (MBP10), and HS014. MT II has the structure Ac-Nle⁴-Asp⁵-His6-D-Phe⁷-Arg⁸-Trp⁹-Lys¹⁰-α-MSH(4-10)-NH₂. PT141 has the structure Ac-Nle-Asp-His-DPhe-Arg-Trp-Lys-OH. HS014 has the structure cyclic [AcCys¹¹, D-Nal¹⁴, Cys¹⁸, Asp-NH₂²²]-β-MSH(11-22) (as described in, for example, Kask et al., Biochem. Biophys. Research Comm 245, 90-93 (1998)).

Other embodiments of the present invention include those in which the second compound is selected from the group consisting of a γ-amino butyric acid (GABA) inhibitor, a GABA receptor antagonist, and a GABA channel antagonist. By “GABA inhibitor” it is meant a compound that reduces the production of GABA in the cells, reduces the release of GABA from the cells, or reduces the activity of GABA on its receptors, either by preventing the binding of GABA to GABA receptors or by minimizing the effect of such binding. The GABA inhibitor may be a 5-HT1b agonist or another agent that inhibits the activity of NPY/AgRP/GABA neurons. In addition, the GABA inhibitor may suppress the expression of the AgRP gene, or the GABA inhibitor may suppress the production or release of AgRP. It is, however, understood that a 5-HT1b agonist may inhibit the NPY/AgRP/GABA neuron (and therefore activate POMC neurons) without acting as an inhibitor of the GABA pathway.

In certain other embodiments the GABA inhibitor increases the expression of the POMC gene. In some of these embodiments, the GABA inhibitor increases the production or release of pro-opiomelanocortin (POMC) protein. In certain other of these embodiments, the GABA inhibitor increases the activity on POMC expressing neurons. In some embodiments, the GABA inhibitor is topiramate.

In other embodiments the second compound is a dopamine reuptake inhibitor. Phentermine is an example of a dopamine reuptake inhibitor. In certain other embodiments, the second compound is a norepinephrine reuptake inhibitor. Examples of norepinephrine reuptake inhibitors include thionisoxetine, and reboxetine. Other embodiments include those in which the second compound is a dopamine agonist. Some dopamine agonists that are available on the market include cabergoline, amantadine, lisuride, pergolide, ropinirole, pramipexole, and bromocriptine. In further embodiments, the second compound is a norepinephrine releaser, for example diethylpropion, or a mixed dopamine/norepinephrine reuptake inhibitor, for example, atomoxatine.

In certain other embodiments, the second compound is a 5-HT1b agonist, such as sumatriptan, almotriptan, naratriptan, frovatriptan, rizatriptan, zomitriptan, and elitriptan.

In further embodiments, the second compound is an anticonvulsant. The anticonvulsant may be selected from the group consisting of zonisamide, topiramate, nembutal, lorazepam, clonazepam, clorazepate, tiagabine, gabapentin, fosphenytoin, phenytoin, carbamazepine, valproate, felbamate, levetiracetam, oxcarbazepine, lamotrigine, methsuximide, and ethosuxmide.

In some embodiments, the second compound is a cannabinoid receptor antagonist. Examples of this group of compounds include AM251 [N-(piperidin-1-yl)-1-(2,4-dichlorophenyl)-5-(4-iodophenyl)-4-methyl-1H-pyrazole-3-carboxamide], AM281 [N-(morpholin-1-yl)-1-(2,4-dichlorophenyl)-5-(4-iodophenyl)-4-methyl-1H-pyrazole-3-carboxamide], AM630 (6-iodo-2-methyl-1-(2-(4-morpholinyl)ethyl]-1H-indol-3-yl](4-methoxyphenyl)methanone), LY320135, and SR141716A (rimonabant), and a pharmaceutically acceptable salt or prodrug thereof. LY320135 and SR141716A have the following structures.

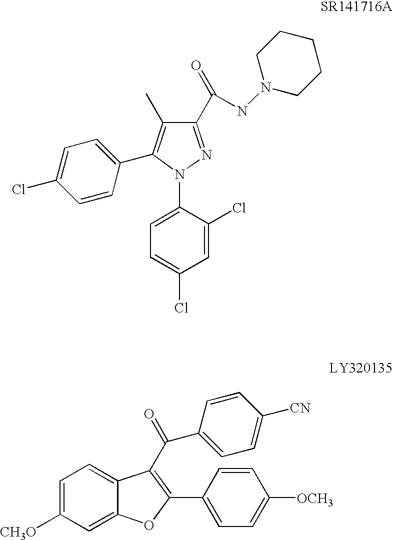

In certain embodiments, the present invention relates to a combination of bupropion and rimonabant. In other embodiments, the present invention relates to a combination of radafaxine and rimonabant.

In some embodiments, the second compound is an agent useful in the treatment of bipolar disorders, which is selected from the group consisting of lithium, valproic acid, valproate, divalproex, carbamezepine, oxycarbamezepine, lamotrogine, tiagabine, and benzodiazepines. In certain embodiments, the second compound is selected from the group consisting of valproic acid, valproate, and divalproex. Divalproex sodium is marketed as DEPAKOTE® by Abbot Laboratories.

In certain embodiments, the present invention relates to a combination of bupropion and divalproex. In other embodiments, the present invention relates to a combination of radafaxine and divalproex.

In certain embodiments, the second compound itself may be a combination of two or more compounds. For example, the second compound may be a combination of a dopamine reuptake inhibitor and a norepinephrine reuptake inhibitor, e.g. mazindol. Alternatively, the second compound may be a combination of a SSRI and a norepinephrine reuptake inhibitor, such as sibutramine, venlafaxine, and duloxetine.

In certain embodiments, the second compound is an activator of the POMC neurons. Examples of POMC activators include Ptx1, leukemia inhibitory factor (LIF), and interleukin 1 beta, (IL-1β).

In certain embodiments, the present invention relates to a combination of bupropion and olanzapine. In other embodiments, the present invention relates to a combination of bupropion and Zyprexa®. Further embodiments relate to a combination of radafaxine and olanzapine, or to a combinatione of radafaxine and Zyperxa®.

In certain embodiments, the compositions of the present invention comprise a third compound, where the third compound is selected from the group of compounds described above for the second compound. In some embodiments, the composition of the invention comprises bupropion, zonisamide, and Zyprexa®. In other embodiments, the composition of the invention comprises radafaxine, zonisamide, and Zyprexa®.

In another aspect, the present invention relates to a method of affecting weight loss, comprising identifying an individual in need thereof and treating that individual with a combination of bupropion, or a metabolite thereof, and a compound that enhances α-MSH activity.

In another aspect, the present invention relates to a method of affecting weight loss, comprising identifying an individual in need thereof and treating that individual with a combination of bupropion, or a metabolite thereof, and a compound that antagonizes cannabinoid receptor activity.

In yet another aspect, the present invention is directed to a method of affecting weight loss, comprising identifying an individual in need thereof and treating that individual with a combination of bupropion, or a metabolite thereof, and a compound that is an agent useful in the treatment of bipolar disorders.

In certain embodiments, the individual has a body mass index (BMI) greater than 25. In other embodiments, the individual has a BMI greater than 30. In still other embodiments, the individual has a BMI greater than 40. However, in some embodiments, the individual may have a BMI less than 25. In these embodiments, it may be beneficial for health or cosmetic purposes to affect weight loss, thereby reducing the BMI even further.

In some of the embodiments set forth above, the compound that enhances α-MSH activity does so by triggering the release of α-MSH or increasing the activity of neurons that express α-MSH. In some embodiments, the compound is a selective serotonin reuptake inhibitor (SSRI) or a specific 5-HT receptor agonist. Examples of SSRIs that can be used in the present invention include fluoxetine, fluvoxamine, sertraline, paroxetine, citalopram, escitalopram, sibutramine, duloxetine, and venlafaxine, and a pharmaceutically acceptable salt or prodrug thereof.

In other embodiments, the compound is a γ-amino butyric acid (GABA) inhibitor. The GABA inhibitor may be a 5-HT1b receptor agonist. The GABA inhibitor may suppress the expression of the AgRP gene, or it may suppresses the production or release of AgRP. The GABA inhibitor may suppress the expression or release of NPY. In certain embodiments, the GABA inhibitor suppresses the activity of neurons that express AgRP. For example, the GABA inhibitor may be topiramate, 1-(2-(((diphenylmethylene)amino)oxy)ethyl)-1,2,5,6-tetrahydro-3-pyridinecarboxylic acid hydrochloride (NNC-711), or vigabatrin.

In certain embodiments, the method of invention set forth above is practiced with the proviso that the individual is not suffering from Prader-Willi syndrome or binge eating disorder. Thus, some embodiments of the invention are to be distinguished from combination therapy involving SSRI anti-depressants (e.g., fluoxetine) used to treat physiological eating disorders such as binge eating disorder or Prader-Willi syndrome. In these embodiments, the target population is the population of individuals needing or desiring weight loss, apart from needing treatment for Prader-Willi syndrome or binge eating disorder.

Individuals suffering from depression may gain weight as a result of their depression. In addition, certain depressed individuals gain weight as a side effect of the depression therapy. In certain embodiments, the method of invention set forth above is practiced with the proviso that the individual is not suffering from depression. In some embodiments, the individual's overweight state was not caused by treatment for depression.

In some embodiments, the treating step of the above method comprises administering to the individual a combination of bupropion, or a metabolite thereof, and a second compound, where the second compound enhances α-MSH activity.

In some embodiments, the treating step of the above method comprises administering to the individual a combination of bupropion, or a metabolite thereof, and a second compound, where the second compound antagonizes cannabinoid receptor activity.

In some embodiments, the treating step of the above method comprises administering to the individual a combination of bupropion, or a metabolite thereof, and a second compound, where the second compound is an agent useful in the treatment of bipolar disorders.

In some embodiments bupropion, or a metabolite thereof, and the second compound are administered more or less simultaneously. In other embodiments bupropion, or a metabolite thereof, is administered prior to the second compound. In yet other embodiments, bupropion, or a metabolite thereof, is administered subsequent to the second compound.

In certain embodiments, bupropion, or a metabolite thereof, and the second compound are administered individually. In other embodiments, bupropion, or a metabolite thereof, and the second compound are covalently linked to each other such that they form a single chemical entity. The single chemical entity is then digested and is metabolized into two separate physiologically active chemical entities, one of which is bupropion, or a metabolite thereof, or a pharmaceutically acceptable salt or prodrug thereof, and the other one is the second compound.

In some embodiments, the compositions of the present invention are a combination of bupropion, or a metabolite thereof, and one or more of the following compounds: a SSRI, a dopamine reuptake inhibitor, a dopamine/norepinephrine reuptake inhibitor, a norepinephrine reuptake inhibitor, an opioid antagonist, a partial opioid agonist, GABA inhibitor, a peripherally acting weight loss agent such as metformin, a peptide, such as PYY, PYY₃₋₃₆, or leptin, a cannabinoid receptor antagonist, and an NPY receptor antagonist, e.g., an NPY Y5 receptor antagonist, such as S-2367.

Examples of norepinephrine agonists include phendimetrazine and benzphetamine. Examples of adenosine compounds include all xanthine derivatives, such as adenosine, caffeine, theophylline, theobromine, and aminophylline. An example of a cholinergic receptor antagonist is nicotine.

In another aspect, the present invention relates to a method of increasing satiety in an individual comprising identifying an individual in need thereof and treating that individual with a combination of bupropion, or a metabolite thereof, and a compound that enhances α-MSH activity.

In another aspect, the present invention relates to a method of increasing satiety in an individual comprising identifying an individual in need thereof and treating that individual with a combination of bupropion, or a metabolite thereof, and a compound that antagonizes cannabinoid receptor activity.

In yet another aspect, the present invention is directed to a method of increasing satiety in an individual, comprising identifying an individual in need thereof and treating that individual with a combination of bupropion, or a metabolite thereof, and a compound that is an agent useful in the treatment of bipolar disorders.

In some embodiments, the treating step of the above method comprises administering to the individual bupropion, or a metabolite thereof, and a second compound, where the second compound enhances α-MSH activity.

In some embodiments bupropion, or a metabolite thereof, and the second compound are administered nearly simultaneously. In other embodiments bupropion, or a metabolite thereof, is administered prior to the second compound. In yet other embodiments, bupropion, or a metabolite thereof, is administered subsequent to the second compound.

In yet another aspect, the present invention relates to a method of suppressing the appetite of an individual comprising identifying an individual in need thereof and treating that individual with a combination of bupropion, or a metabolite thereof, and a compound that enhances α-MSH activity.

In yet another aspect, the present invention relates to a method of suppressing the appetite of an individual comprising identifying an individual in need thereof and treating that individual with a combination of bupropion, or a metabolite thereof, and a compound that antagonizes cannabinoid receptor activity.

In yet another aspect, the present invention is directed to a method of suppressing the appetite of an individual, comprising identifying an individual in need thereof and treating that individual with a combination of bupropion, or a metabolite thereof, and a compound that is an agent useful in the treatment of bipolar disorders.

In some embodiments, the treating step of the above method comprises administering to the individual bupropion, or a metabolite thereof, and a second compound, where the second compound enhances α-MSH activity.

In some embodiments bupropion, or a metabolite thereof, and the second compound are administered nearly simultaneously. In other embodiments bupropion, or a metabolite thereof, is administered prior to the second compound. In yet other embodiments, bupropion, or a metabolite thereof, is administered subsequent to the second compound.

In another aspect, the present invention relates to a method of increasing energy expenditure in an individual comprising identifying an individual in need thereof and treating that individual with a combination of bupropion, or a metabolite thereof, and a compound that enhances α-MSH activity.

In another aspect, the present invention relates to a method of increasing energy expenditure in an individual comprising identifying an individual in need thereof and treating that individual with a combination of bupropion, or a metabolite thereof, and a compound that antagonizes cannabinoid receptor activity.

In yet another aspect, the present invention is directed to a method of increasing energy expenditure in an individual, comprising identifying an individual in need thereof and treating that individual with a combination of bupropion, or a metabolite thereof, and a compound that is an agent useful in the treatment of bipolar disorders.

In some embodiments, the treating step of the above method comprises administering to the individual bupropion, or a metabolite thereof, and a second compound, where the second compound enhances α-MSH activity.

In some embodiments bupropion, or a metabolite thereof, and the second compound are administered nearly simultaneously. In other embodiments bupropion, or a metabolite thereof, is administered prior to the second compound. In yet other embodiments, bupropion, or a metabolite thereof, is administered subsequent to the second compound.

In certain embodiments, in the compositions or methods disclosed herein, the second compound does not trigger the release of α-melanocyte stimulating hormone (α-MSH). In some embodiments, the second compound does not increase the extracellular serotonin concentrations in the hypothalamus. In further embodiments, the second compound is not a selective serotonin reuptake inhibitor (SSRI), is not a serotonin 2C agonist, or is not a serotonin 1B agonist. In some embodiments, the second compound is not fluoxetine, is not fluvoxamine, is not sertraline, is not paroxetine, is not citalopram, is not escitalopram, is not sibutramine, is not duloxetine, or is not venlafaxine.

In certain embodiments, in the compositions or methods disclosed herein, the second compound does not suppress the expression of the AgRP gene or the production or release of agouti-related protein (AgRP). In some of these embodiments, the second compound does not suppress the activity of neurons that express AgRP.

In other embodiments, in the compositions or methods disclosed herein, the second compound does not suppress the expression of the NPY gene or the production or release of neuropeptide Y (NPY). In some of these embodiments, the second compound does not suppress the activity of neurons that express NPY. In further embodiments, the second compound is not an NPY antagonist, is not a ghrelin antagonist, or is not leptin. In certain other embodiments, the second compound does not agonize NPY Y2 receptor.

In some embodiments, in the compositions or methods disclosed herein, the second compound is not an NPY receptor antagonist. In certain embodiments, the second compound is not an NPY Y1 receptor antagonist, while in other embodiments the second compound is not an NPY Y5receptor antagonist. In some embodiments, the NPY receptor antagonist is not S-2367.

In other embodiments, in the compositions or methods disclosed herein, the second compound is not a GABA inhibitor, is not a GABA receptor antagonist, or is not a GABA channel antagonist.

In certain other embodiments, in the compositions or methods disclosed herein, the GABA inhibitor does not increase the expression of the POMC gene. In some of these embodiments, the GABA inhibitor does not increase the production or release of POMC protein. In certain other of these embodiments, the GABA inhibitor does not increase the activity on POMC expressing neurons. In some embodiments, the GABA inhibitor is not topiramate.

In other embodiments, in the compositions or methods disclosed herein, the second compound is not a dopamine reuptake inhibitor. In other embodiments, the dopamine reuptake inhibitor is not phentermine. In certain other embodiments, the second compound is not a norepinephrine reuptake inhibitor. In other embodiments, the norepinephrine reuptake inhibitor is not thionisoxetine or is not reboxetine. In further embodiments, the second compound is not a dopamine agonist. In some embodiments, the dopamine agonist is not cabergoline, is not amantadine, is not lisuride, is not pergolide, is not ropinirole, is not pramipexole, or is not bromocriptine. In further embodiments, the second compound is not a norepinephrine releaser. In some embodiments, the norepinephrine releaser is not diethylpropion. In certain embodiments, the second compound is not a mixed dopamine/norepinephrine reuptake inhibitor. In some embodiments, the mixed dopamine/norepinephrine reuptake inhibitor is not atomoxatine.

In certain other embodiments, in the compositions or methods disclosed herein, the second compound is not a 5-HT1b agonist. In some embodiments, the 5-HT1b agonist is not sumatriptan, is not almotriptan, is not naratriptan, is not frovatriptan, is not rizatriptan, is not zomitriptan, or is not elitriptan.

In further embodiments, in the compositions or methods disclosed herein, the second compound is not an anticonvulsant. In some embodiments, the anticonvulsant is not zonisamide, is not topiramate, is not nembutal, is not lorazepam, is not clonazepam, is not clorazepate, is not tiagabine, is not gabapentin, is not fosphenytoin, is not phenytoin, is not carbamazepine, is not valproate, is not felbamate, is not levetiracetam, is not oxcarbazepine, is not lamotrigine, is not methsuximide, or is not ethosuxmide.

In some embodiments, in the compositions or methods disclosed herein, the second compound is not a cannabinoid receptor antagonist. In some embodiments, cannabinoid receptor antagonist is not AM251, is not AM281, is not AM630, is not LY320135, or is not SR141716A.

In certain embodiments, in the compositions or methods disclosed herein, the second compound is not an activator of the POMC neurons. In some embodiments, the activator of the POMC neurons is not Ptx1 or is not IL-1β.

In certain embodiments disclosed herein, an individual is given a pharmaceutical composition comprising a combination of two or more compounds to affect weight loss. In some of these embodiments, each compound is a separate chemical entity. However, in other embodiments, the two compounds are joined together by a chemical linkage, such as a covalent bond, so that the two different compounds form separate parts of the same molecule. The chemical linkage is selected such that after entry into the body, the linkage is broken, such as by enzymatic action, acid hydrolysis, base hydrolysis, or the like, and the two separate compounds are then formed.

Thus, in another aspect, the present invention relates to synthetic routes to novel molecules in which an opioid antagonist is linked by a flexible linker to another compound disclosed herein.

In another aspect, the invention relates to a pharmaceutical composition comprising a combination of bupropion, or a metabolite thereof, and a compound that causes increased agonism of a melanocortin 3 receptor (MC3-R) or a melanocortin 4 receptor (MC4-R) compared to normal physiological conditions, as described above, or comprising a linked molecule, as described herein, and a physiologically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

It will be understood by those of skill in the art that numerous and various modifications can be made without departing from the spirit of the present invention. Therefore, it should be clearly understood that the forms of the present invention are illustrative only and are not intended to limit the scope of the present invention.

### Some Embodiments of the Invention

Some of the embodiments of the present invention are as follows:

In the 1^{st} embodiment, the invention relates to a composition for affecting weight loss comprising bupropion, or a metabolite thereof, and a second compound, wherein said second compound causes increased agonism of a melanocortin 3 receptor (MC3-R) or a melanocortin 4 receptor (MC4-R) compared to normal physiological conditions, or wherein said second compound antagonizes cannabinoid receptor activity.

In the 2^{nd} embodiment, the invention relates to the composition of the 1^{st} embodiment, wherein said second compound triggers the release of α-melanocyte stimulating hormone (α-MSH).

In the 3^{rd} embodiment, the invention relates to the composition of the 2^{nd} embodiment, wherein said second compound increases the extracellular serotonin concentrations in the hypothalamus.

In the 4^{th} embodiment, the invention relates to the composition of the 3^{rd} embodiment, wherein said second compound is selected from the group consisting of a selective serotonin reuptake inhibitor (SSRI), a serotonin 2C agonist, and a serotonin 1B agonist.

In the 5^{th} embodiment, the invention relates to the composition of the 4^{th} embodiment, wherein said second compound is selected from the group consisting of fluoxetine, fluvoxamine, sertraline, paroxetine, citalopram, escitalopram, sibutramine, duloxetine, and venlafaxine, and pharmaceutically acceptable salts or prodrugs thereof.

In the 6^{th} embodiment, the invention relates to the composition of the 1^{st} embodiment, wherein said second compound suppresses the expression of the AgRP gene or the production or release of agouti-related protein (AgRP).

In the 7^{th} embodiment, the invention relates to the composition of the 1^{st} embodiment, wherein said second compound suppresses the activity of neurons that express AgRP.

In the 8^{th} embodiment, the invention relates to the composition of the 1^{st} embodiment, wherein said second compound suppresses the expression of the NPY gene or the production or release of neuropeptide Y (NPY).

In the 9^{th} embodiment, the invention relates to the composition of the 1^{st} embodiment, wherein said second compound suppresses the activity of neurons that express NPY.

In the 10^{th} embodiment, the invention relates to the composition of the 1^{st} embodiment, wherein said second compound is an NPY receptor antagonist.

In the 11^{th} embodiment, the invention relates to the composition of the 10^{th} embodiment, wherein said NPY receptor is selected from NPY Y1 receptor, NPY Y2 receptor, NPY Y4 receptor, and NPY Y5 receptor.

In the 12^{th} embodiment, the invention relates to the composition of the 1^{th} embodiment, wherein said compound is S-2367.

In the 13^{th} embodiment, the invention relates to the composition of the 1^{st} embodiment, wherein said second compound is selected from the group consisting of ghrelin antagonists and leptin.

In the 14^{th} embodiment, the invention relates to the composition of the 1^{st} embodiment, wherein said second compound agonizes NPY Y2 receptor.

In the 15^{th} embodiment, the invention relates to the composition of the 1^{st} embodiment, wherein said second compound is selected from the group consisting of a γ-amino butyric acid (GABA) inhibitor, a GABA receptor antagonist, and a GABA channel antagonist.

In the 16^{th} embodiment, the invention relates to the composition of the 15^{th} embodiment, wherein said GABA inhibitor is a 5-HT1b agonist, which may be selected from sumatriptan, almotriptan, naratriptan, frovatriptan, rizatriptan, zomitriptan, and elitriptan.

In the 17^{th} embodiment, the invention relates to the composition of the 15^{th} embodiment, wherein said GABA inhibitor suppresses the expression of the AgRP gene.

In the 18^{th} embodiment, the invention relates to the composition of the 15^{th} embodiment, wherein said GABA inhibitor suppresses the production or release of AgRP.

In the 19^{th} embodiment, the invention relates to the composition of the 15^{th} embodiment, wherein said GABA inhibitor increases the expression of the POMC gene.

In the 20^{th} embodiment, the invention relates to the composition of the 15^{th} embodiment, wherein said GABA inhibitor increases the production or release of α-MSH from pro-opiomelanocortin (POMC) neurons.

In the 21^{st} embodiment, the invention relates to the composition of the 15^{th} embodiment, wherein said GABA inhibitor increases the activity of POMC expressing neurons.

In the 22^{nd} embodiment, the invention relates to the composition of the 15^{th} embodiment, wherein the GABA inhibitor is topiramate.

In the 23^{rd} embodiment, the invention relates to the composition of the 1^{st} embodiment, wherein said second compound is a dopamine reuptake inhibitor.

In the 24^{th} embodiment, the invention relates to the composition of the 23^{rd} embodiment, wherein said dopamine reuptake inhibitor is phentermine.

In the 25^{th} embodiment, the invention relates to the composition of the 1^{st} embodiment, wherein said second compound is a norepinephrine reuptake inhibitor.

In the 26^{th} embodiment, the invention relates to the composition of the 25^{th} embodiment, wherein said norepinephrine reuptake inhibitor is selected from thionisoxetine and reboxetine.

In the 27^{th} embodiment, the invention relates to the composition of the 1^{st} embodiment, wherein said second compound is a dopamine agonist.

In the 28^{th} embodiment, the invention relates to the composition of the 27^{th} embodiment, wherein said dopamine agonist is selected from the group consisting of cabergoline, amantadine, lisuride, pergolide, ropinirole, pramipexole, and bromocriptine.

In the 29^{th} embodiment, the invention relates to the composition of the 1^{st} embodiment, wherein said second compound is a norepinephrine releaser.

In the 30^{th} embodiment, the invention relates to the composition of the 29^{th} embodiment, wherein said norepinephrine releaser is diethylpropion.

In the 31^{st} embodiment, the invention relates to the composition of the 1^{st} embodiment, wherein said second compound is a combination of a dopamine reuptake inhibitor and a norepinephrine reuptake inhibitor.

In the 32^{nd} embodiment, the invention relates to the composition of the 31^{st} embodiment, wherein said second compound is mazindol.

In the 33^{rd} embodiment, the invention relates to the composition of the 1^{st} embodiment, wherein said second compound is a combination of a SSRI and a norepinephrine reuptake inhibitor.

In the 34^{th} embodiment, the invention relates to the composition of the 33^{rd} embodiment, wherein said second compound is selected from sibutramine, venlafaxine, and duloxetine.

In the 35^{th} embodiment, the invention relates to the composition of the 1^{st} embodiment, wherein said second compound is a cannabinoid receptor antagonist.

In the 36^{th} embodiment, the invention relates to the composition of the 35^{th} embodiment, wherein said cannabinoid receptor antagonist is selected from the group consisting of AM251 [N-(piperidin-1-yl)-1-(2,4-dichlorophenyl)-5-(4-iodophenyl)-4-methyl-1H-pyrazole-3-carboxamide], AM281 [N-(morpholin-1-yl)-1-(2,4-dichlorophenyl)-5-(4-iodophenyl)-4-methyl-1H-pyrazole-3-carboxamide], AM630 (6-iodo-2-methyl-1-[2-(4-morpholinyl)ethyl]-1H-indol-3-yl](4-methoxyphenyl)methanone), LY320135, and SR141716A (rimonabant), and pharmaceutically acceptable salts or prodrugs thereof.

In the 37^{th} embodiment, the invention relates to the composition of the 1^{st} embodiment, wherein said second compound is AM251.

In the 38^{th} embodiment, the invention relates to a method of affecting weight loss, comprising identifying an individual in need thereof and treating that individual with a combination of bupropion, or a metabolite thereof, and a compound that enhances α-MSH activity or antagonizes cannabinoid receptor activity.

In the 39^{th} embodiment, the invention relates to the method of the 38^{th} embodiment, wherein said individual has a body mass index greater than 25.

In the 40^{th} embodiment, the invention relates to the method of the 38^{th} embodiment, wherein α-MSH activity is enhanced by administering a compound, wherein said compound triggers release of α-MSH or increases the activity of neurons that express α-MSH.

In the 41^{st} embodiment, the invention relates to the method of the 40^{th} embodiment, wherein said compound is a selective serotonin reuptake inhibitor (SSRI) or a specific 5-HT receptor agonist.

In the 42^{nd} embodiment, the invention relates to the method of the 41^{st} embodiment, wherein said 5-HT receptor is selected from 5-HT1b receptor and 5-HT2c receptor.

In the 43^{rd} embodiment, the invention relates to the method of the 41^{st} embodiment, wherein said SSRI is selected from fluoxetine, fluvoxamine, sertraline, paroxetine, citalopram, escitalopram, sibutramine, duloxetine, and venlafaxine, and pharmaceutically acceptable salts or prodrugs thereof.

In the 44^{th} embodiment, the invention relates to the method of the 40^{th} embodiment, wherein said compound is a γ-amino butyric acid (GABA) inhibitor.

In the 45^{th} embodiment, the invention relates to the method of the 44^{th} embodiment, wherein said GABA inhibitor is a 5-HT1b receptor agonist.

In the 46^{th} embodiment, the invention relates to the method of the 44^{th} embodiment, wherein said GABA inhibitor suppresses the expression of the AgRP gene.

In the 47^{th} embodiment, the invention relates to the method of the 44^{th} embodiment, wherein said GABA inhibitor suppresses the production or release of AgRP.

In the 48^{th} embodiment, the invention relates to the method of the 41^{st} embodiment, wherein said 5-HT agonists inhibits the NPY/AgRP/GABA neurons.

In the 49^{th} embodiment, the invention relates to the method of the 38^{th} embodiment, wherein said second compound suppresses the activity of neurons that express NPY.

In the 50^{th} embodiment, the invention relates to the method of the 38^{th} embodiment, wherein said second compound is an NPY receptor antagonist.

In the 51^{st} embodiment, the invention relates to the method of the 50^{th} embodiment, wherein said NPY receptor is selected from NPY Y1 receptor, NPY Y2 receptor, NPY Y4 receptor, and NPY Y5 receptor.

In the 52^{nd} embodiment, the invention relates to the method of the 51^{st} embodiment, wherein said compound is S-2367.

In the 53^{rd} embodiment, the invention relates to the method of the 44^{th} embodiment, wherein said GABA inhibitor suppresses the activity of neurons that express AgRP.

In the 54^{th} embodiment, the invention relates to the method of the 44^{th} embodiment, wherein said GABA inhibitor is topiramate.

In the 55^{th} embodiment, the invention relates to the method of the 40^{th} embodiment, wherein said compound is selected from the group consisting of a dopamine reuptake inhibitor, a norepinephrine reuptake inhibitor, a dopamine agonist, a norepinephrine releaser, a combination of a dopamine reuptake inhibitor and a norepinephrine reuptake inhibitor, and a combination of a SSRI and a norepinephrine reuptake inhibitor.

In the 56^{th} embodiment, the invention relates to the method of the 38^{th} embodiment, wherein said treating step comprises administering to said individual bupropion, or a metabolite thereof, and a second compound, wherein said second compound enhances α-MSH activity, or wherein said second compound antagonizes cannabinoid receptor activity.

In the 57^{th} embodiment, the invention relates to the method of the 56^{th} embodiment, wherein bupropion, or a metabolite thereof, and said second compound are administered nearly simultaneously.

In the 58^{th} embodiment, the invention relates to the method of the 57^{th} embodiment, wherein bupropion, or a metabolite thereof, is administered prior to said second compound.

In the 59^{th} embodiment, the invention relates to the method of the 58^{th} embodiment, wherein bupropion, or a metabolite thereof, is administered subsequent to said second compound.

In the 60^{th} embodiment, the invention relates to a method of increasing satiety in an individual comprising identifying an individual in need thereof and treating that individual with a combination of bupropion, or a metabolite thereof, and a compound that enhances α-MSH activity or antagonizes cannabinoid receptor activity.

In the 61^{st} embodiment, the invention relates to the method of the 60^{th} embodiment, wherein said treating step comprises administering to said individual bupropion, or a metabolite thereof, and a second compound, wherein said second compound enhances α-MSH activity or antagonizes cannabinoid receptor activity.

In the 62^{nd} embodiment, the invention relates to the method of the 61^{st} embodiment, wherein bupropion, or a metabolite thereof, and said second compound are administered nearly simultaneously.

In the 63^{rd} embodiment, the invention relates to the method of the 61^{st} embodiment, wherein bupropion, or a metabolite thereof, is administered prior to said second compound.

In the 64^{th} embodiment, the invention relates to the method of the 61^{st} embodiment, wherein bupropion, or a metabolite thereof, is administered subsequent to said second compound.

In the 65^{th} embodiment, the invention relates to a method of increasing energy expenditure in an individual comprising identifying an individual in need thereof and treating that individual with a combination of bupropion, or a metabolite thereof, and a compound that enhances α-MSH activity, or antagonizes cannabinoid receptor activity.

In the 66^{th} embodiment, the invention relates to the method of the 65^{th} embodiment, wherein said treating step comprises administering to said individual bupropion, or a metabolite thereof, and a second compound, wherein said second compound enhances α-MSH activity or antagonizes cannabinoid receptor activity.

In the 67^{th} embodiment, the invention relates to the method of the 66^{th} embodiment, wherein bupropion, or a metabolite thereof, and said second compound are administered nearly simultaneously.

In the 68^{th} embodiment, the invention relates to the method of the 66^{th} embodiment, wherein bupropion, or a metabolite thereof, is administered prior to said second compound.

In the 69^{th} embodiment, the invention relates to the method of the 66^{th} embodiment, wherein bupropion, or a metabolite thereof, is administered subsequent to said second compound.

In the 70^{th} embodiment, the invention relates to a method of suppressing the appetite of an individual comprising identifying an individual in need thereof and treating that individual with a combination of bupropion, or a metabolite thereof, and a compound that enhances α-MSH activity, or antagonizes cannabinoid receptor activity.

In the 71^{st} embodiment, the invention relates to the method of the 70^{th} embodiment, wherein said treating step comprises administering to said individual bupropion, or a metabolite thereof, and a second compound, wherein said second compound enhances α-MSH activity, or antagonizes cannabinoid receptor activity.

In the 72^{nd} embodiment, the invention relates to the method of the 71^{st} embodiment, wherein bupropion, or a metabolite thereof, and said second compound are administered nearly simultaneously.

In the 73^{rd} embodiment, the invention relates to the method of the 71^{st} embodiment, wherein bupropion, or a metabolite thereof, is administered prior to said second compound.

In the 74^{th} embodiment, the invention relates to the method of the 71^{st} embodiment, wherein bupropion, or a metabolite thereof, is administered subsequent to said second compound.

In the 75^{th} embodiment, the invention relates to the method of the 38^{th} embodiment through the 74^{th} embodiment, wherein the individual has a BMI greater than 30.

In the 76^{th} embodiment, the invention relates to the method of the 75^{th} embodiment, wherein the individual has a BMI greater than 25.

### EXAMPLES

The examples below are non-limiting and are merely representative of various aspects of the invention.

### Example 1

### Combination of Fluoxetine and Bupropion

Individuals having a BMI of greater than 25 are identified. Each individual is instructed to take one 20 mg tablet of fluoxetine (PROZAC®) on a daily basis, in addition to one 75 mg tablet of bupropion on a daily basis. The administered bupropion may be in a sustained release formulation.

The individuals are monitored for a period of months. It is recommended that the dosage be adjusted so that each individual loses weight at a rate of 10% of initial weight every 6 months. However, the rate of weigh loss for each individual may be adjusted by the treating physician based on the individual's particular needs.

If the initial dosage is not effective, then the fluoxetine dosage can be increased by 20 mg per day, though never exceeding 80 mg total per day. The bupropion dosage can be increased to 100 or 150 mg on a daily basis. If the initial dosage results in a more rapid weight loss than the above rate, the dosage of each of fluoxetine or bupropion can be reduced.

### Example 2

### Combination of Bupropion and Sibutramine

Individuals having a BMI of greater than 25 are identified. Each individual is instructed to take bupropion in the dosage set forth in Example 1. In addition, each individual is instructed to take 10 mg of sibutramine orally once a day.

The individuals are monitored for a period of months. It is recommended that the dosage be adjusted so that each individual loses weight at a rate of 10% of initial weight every 6 months. However, the rate of weigh loss for each individual may be adjusted by the treating physician based on the individual's particular needs.

If the initial dosage is not effective, then the sibutramine dosage can be increased 15 mg per day. Dosages of sibutramine in excess of 15 mg per day are not recommended. The bupropion dosage can be increased to 100 or 150 mg on a daily basis. If the initial dosage results in a more rapid weight loss than the above rate, the dosage of each of sibutramine or bupropion can be reduced.

### Example 3

### Combination of Opioid Antagonist and Phentermine

Individuals having a BMI of greater than 25 are identified. Each individual is instructed to take bupropion in the dosage set forth in Example 1. In addition, each individual is instructed to take 37.5 mg of phentermine orally once a day.

The individuals are monitored for a period of months. It is recommended that the dosage be adjusted so that each individual loses weight at a rate of 10% of initial weight every 6 months. However, the rate of weigh loss for each individual may be adjusted by the treating physician based on the individual's particular needs.

### Example 4

### Combination of AM251 and Bupropion

Individuals having a BMI of greater than 25 are identified. Each individual is instructed to take one 20 mg tablet of AM251 on a daily basis. In addition, each individual is instructed to take bupropion in the dosage set forth in Example 1.

The individuals are monitored for a period of months. It is recommended that the dosage be adjusted so that each individual loses weight at a rate of 10% of initial weight every 6 months. However, the rate of weigh loss for each individual may be adjusted by the treating physician based on the individual's particular needs.

If the initial dosage is not effective, then the AM251 dosage can be increased by 20 mg per day, though never exceeding 80 mg total per day. The bupropion dosage can be increased to 100 or 150 mg on a daily basis. If the initial dosage results in a more rapid weight loss than the above rate, the dosage of each of AM251 or bupropion can be reduced.

In some cases, it is beneficial to administer one dose of AM251 per day in conjunction with two or three or more doses of naltrexone throughout the day. Naltrexone may also be in a time-release formulation where the dose is administered once a day, but naltrexone gradually enters the blood stream throughout the day, or in the course of a 12 hour period.

### Example 5

### Combination of Bupropion and DEPAKOTE®

Individuals having a BMI of greater than 25 are identified. Each individual is instructed to take bupropion in the dosage set forth in Example 1. In addition, each individual is instructed to take 250 mg of DEPAKOTE® orally twice a day.

The individuals are monitored for a period of months. It is recommended that the dosage be adjusted so that each individual loses weight at a rate of 10% of initial weight every 6 months. However, the rate of weigh loss for each individual may be adjusted by the treating physician based on the individual's particular needs.

If the initial dosage is not effective, then the DEPAKOTE® dosage can be increased 500 mg twice a day, then to 1000 mg twice a day, and if still greater weight loss rate is desired, to 1000 mg four times a day. The bupropion dosage can be increased to 100 or 150 mg on a daily basis. If the initial dosage results in a more rapid weight loss than the above rate, the dosage of each of DEPAKOTE® or bupropion can be reduced.

What is claimed is:

1. A composition for affecting weight loss comprising bupropion, or a metabolite thereof, and a second compound, wherein said second compound causes increased agonism of a melanocortin 3 receptor (MC3-R) or a melanocortin 4 receptor (MC4-R) compared to normal physiological conditions, or wherein said second compound antagonizes cannabinoid receptor activity.

2. The composition of claim 1, wherein said second compound is selected from the group consisting of a selective serotonin reuptake inhibitor (SSRI), a serotonin 2C agonist, and a serotonin 1B agonist.

3. The composition of claim 2, wherein said second compound is selected from the group consisting of fluoxetine, fluvoxamine, sertraline, paroxetine, citalopram, escitalopram, sibutramine, duloxetine, and venlafaxine, and pharmaceutically acceptable salts or prodrugs thereof.

4. The composition of claim 2, wherein said second compound is sibutramine.

5. The composition of claim 1, wherein said metabolite of bupropion is radafaxine.

6. The composition of claim 1, wherein said second compound is a dopamine reuptake inhibitor.

7. The composition of claim 6, wherein said dopamine reuptake inhibitor is phentermine.

8. The composition of claim 1, wherein said second compound is a cannabinoid receptor antagonist.

9. The composition of claim 8, wherein said cannabinoid receptor antagonist is selected from the group consisting of AM251 [N-(piperidin-1-yl)-1-(2,4-dichlorophenyl)-5-(4-iodophenyl)-4-methyl-1H-pyrazole-3-carboxamide], AM281 [N-(morpholin-1-yl)-1-(2,4-dichlorophenyl)-5-(4-iodophenyl)-4-methyl-1H-pyrazole-3-carboxamide], AM630 (6-iodo-2-methyl-1-[2-(4-morpholinyl)ethyl]-H-indol-3-yl](4-methoxyphenyl)methanone), LY320 135, and SR141716A (rimonabant), and pharmaceutically acceptable salts or prodrugs thereof.

10. The composition of claim 8, wherein said cannabinoid receptor antagonist is SR141716A (rimonabant).

11. The composition of claim 8, wherein said second compound is AM251.

12. A composition for the treatment of obesity or for affecting weight loss comprising bupropion, or a metabolite thereof, or a pharmaceutically acceptable salt or prodrug thereof, and a second compound, where the second compound is an agent useful in the treatment of bipolar disorders.

13. The composition of claim 12, wherein said metabolite of bupropion is radafaxine.

14. The composition of claim 12, wherein said agent useful in the treatment of bipolar disorders is selected from the group consisting of lithium, valproic acid, valproate, divalproex, carbamezepine, oxycarbamezepine, lamotrogine, tiagabine, and benzodiazepines.

15. The composition of claim 12, wherein said agent useful in the treatment of bipolar disorders is selected from the group consisting of valproic acid, valproate, and divalproex.

16. A method of affecting weight loss, comprising identifying an individual in need thereof and treating that individual with a combination of bupropion, or a metabolite thereof, and a compound that enhances α-MSH activity, antagonizes cannabinoid receptor activity, or is useful in the treatment of bipolar disorders.

17. The method of claim 16, wherein said individual has a body mass index greater than 25.

18. The method of claim 16, wherein said metabolite of bupropion is radafaxine.

19. The method of claim 16, wherein said compound that enhances α-MSH activity is selected from the group consisting of phentermine and sibutramine.

20. The method of claim 16, wherein said cannabinoid receptor antagonist is selected from the group consisting of SR141716A (rimonabant) and AM251.

21. The method of claim 16, wherein said compound useful in the treatment of bipolar disorders is selected from the group consisting of valproic acid, valproate, and divalproex.
